(19) United States
(12) Patent Application Publication
(10) Pub. No.: US 2017/0238852 A1
(43) Pub. Date: Aug. 24, 2017

(54) EXTENDED ANALYTICAL PERFORMANCE OF CONTINUOUS GLUCOSE MONITORING DEVICES VIA NITRIC OXIDE

(71) Applicant: THE UNIVERSITY OF NORTH CAROLINA AT CHAPEL HILL, Chapel Hill, NC (US)

(72) Inventors: Mark H. Schoenfisch, Chapel Hill, NC (US); Robert Soto, Durham, NC (US)

(21) Appl. No.: 15/321,600

(22) PCT Filed: Jun. 21, 2015
(86) PCT No.: PCT/US15/36852

§ 371 Date: Dec. 22, 2016

Related U.S. Application Data
(60) Provisional application No. 62/015,508, filed on Jun. 22, 2014.

(51) Int. Cl. A61B 5/145; A61B 5/00; A61B 5/1495

(52) U.S. Cl. CPC A61B 5/14503; A61B 5/14532; A61B 5/14546; A61B 5/1495; A61B 5/6848; A61B 5/686

(57) ABSTRACT

The present invention relates to instruments and methods related to the in vivo analytical performance of percutaneously implanted nitric oxide (NO)-releasing amperometric glucose biosensors. Needle-type glucose biosensors can be functionalized with NO-releasing polyurethane coatings designed to release similar total amounts of NO for rapid or slower (greater than 3 day) durations and remain functional as outer glucose sensor membranes. Relative to controls, NO-releasing sensors were characterized with improved numerical accuracy on days 1 and 3. Furthermore, the clinical accuracy and sensitivity of rapid NO-releasing sensors were superior to control and slower NO-releasing sensors at both 1 and 3 days implantation. In contrast, the slower, extended NO releasing-sensors were characterized by shorter sensor lag times (<4.2 min) in response to intravascular glucose tolerance tests versus burst NO-releasing and control sensors (>5.8 min) at 3, 7, and 10 d. Collectively, these results highlight the potential for NO release to enhance the analytical utility of in vivo glucose biosensors. Thus, the analytical performance benefit is dependent on the NO-release duration.

The present application claims priority under 35 USC 119(e) to U.S. Provisional Application No. 62/015,508 filed Jun. 22, 2014, the entire contents of which is incorporated by reference in its entirety.

The present invention is supported at least in part by the National Institutes of Health Grant Numbers R01 EB000708 and R43DK093119. Thus, the Federal Government has rights in the present invention.

### BACKGROUND OF THE INVENTION

Diabetes mellitus is a worldwide epidemic characterized by chronic hyperglycemia that results from either a deficiency or tolerance in insulin. In the United States, 8.3% of the population currently has diabetes and that number is projected to increase to 1 in 3 adults by 2050 if current trends continue. Blood glucose levels in diabetics fluctuate significantly throughout the day, resulting in serious complications including heart attacks, strokes, high blood pressure, kidney failure, blindness and limb amputation. Portable glucose sensors give patients the ability to monitor blood glucose levels, manage insulin levels, and reduce the morbidity and mortality of diabetes mellitus.

Despite the obvious benefits of continuous glucose monitoring (CGM) for the management of diabetes, the utility of in vivo amperometric glucose biosensors is limited to ≦1 week due to poor analytical performance, resulting primarily from the foreign body response (FBR). Insertion of the sensor damages vascularized tissue and results in a cascade of inflammatory events, many of which negatively impact glucose measurements. For example, the resulting passive adsorption of biomolecules (mainly <15 kDa protein fragments) to the sensor surface initiates an inflammatory response and is responsible for a dramatic decrease in sensor sensitivity (˜50%) following sensor implantation. Increased metabolic activity of inflammatory cells (e.g., macrophages and foreign body giant cells) at the sensor-tissue interface results in inordinate consumption of glucose and oxygen, decreasing their local concentrations and attenuating sensor performance. The hallmark of the FBR is the formation of a thick, avascular collagen capsule surrounding the sensor, isolating it from the surrounding tissue and obstructing mass transport of interstitial glucose to the sensor. Indeed, the FBR increases sensor response time, decreases sensitivity, and often results in device failure.

Efforts to improve the analytical performance of in vivo biosensors have largely focused on chemical or physical modifications to the outermost, tissue-contacting membrane to mitigate the FBR. Examples of such strategies include biomimicry (e.g., the attachment of phospholipids to coating surfaces), employing naturally-derived materials as coatings, utilizing membranes that reduce cell adhesion, encouraging tissue ingrowth into porous coatings, and modulating cell behavior through coating topography. The active release of anti-inflammatory or pro-angiogenic bioactive agents such as dexamethasone (DX) and vascular endothelial growth factor (VEGF) has also been proposed as a viable option for improving glucose sensor function. However, in addition to the immune suppression associated with DX and pro-inflammatory roles of VEGF, the controlled release of these molecules from sensor coatings remains a major hurdle.

The inventors have examined the FBR to subcutaneously implanted NO-releasing xerogels coated on silicone elastomers in a murine model. Nitric oxide-releasing implants, which generated ˜1.35 μmol cm⁻² NO over 72 h at fluxes >1 pmol cm⁻²s⁻¹, elicited only a mild FBR with reduced fibrous encapsulation (>25%) after 3 and 6 w compared to tissue near control implants. Concomitant with a reduced FBR, blood vessel density in the tissue surrounding the NO-releasing implants was greater (˜50%) than that observed surrounding control implants. The inventors have also assessed glucose recovery as a function of NO release percutaneously implanted microdialysis probes. A constant NO flux (162 pmol cm⁻² s⁻¹, 4.6 μmol cm⁻² NO daily) was achieved from microdialysis probes by using a saturated NO solution as the perfusate. While glucose recovery from control probes was severely diminished beyond 7 d, NO-releasing microdialysis probes exhibited near constant glucose recovery throughout the study. These results were correlated to tissue histology observations. Indeed, histological analysis of the tissue surrounding NO-releasing probes at 14 d revealed lower inflammatory cell counts and a thinner collagen capsule versus probes that did not release NO.

The lessened FBR and increased glucose recovery suggest that NO release lowered tissue impedance to glucose transport. In a separate study, the inventors have investigated the effects of NO-release kinetics on the FBR to subcutaneous NO-releasing wire implants (i.e., mock glucose sensors) in a porcine model. Decreased collagen capsule thickness (>50%) was observed for substrates that released NO for extended durations (i.e., >14 d) versus wires that did not release NO. In contrast, substrates with shorter NO-release durations (12-24 h) were characterized by greater collagen density at the implant-tissue interface compared to the materials which released NO for extended durations. Collectively, this body of work highlights the dramatic effect of NO-release kinetics on the FBR and the potential to impact the analytical performance of in vivo glucose biosensors.

Despite extensive characterization of the host response to NO-releasing implants, the interplay between reduced FBR and actual sensor performance remains a critical void. To date, only one study has evaluated the in vivo performance of a NO-releasing glucose sensor. Others reported improved clinical accuracy for NO-releasing needle-type glucose biosensors implanted in rats for 3 d. However, the NO release from the sensors was limited to 16 h and deterioration of sensor performance by day 3 was observed. Histological analysis of the surrounding tissues revealed suppressed inflammation at NO-releasing sensors on day 1 versus controls, but no benefits following depletion of the NO reservoir. It is with these deficiencies in mind that the present invention was made.

### BRIEF SUMMARY OF THE INVENTION

The inventors have showed that a controlled release of nitric oxide (NO), an endogenous molecule with multiple roles in inflammation, wound healing, and angiogenesis, from polymeric coatings has shown ability to minimize the foreign body response (FBR). Thus, in one embodiment, the present invention relates to instruments and methods that use NO-releasing glucose monitoring sensors as a means of monitoring glucose levels, including their use for subjects that have or may develop diabetes.

The inventors have discovered that the severity of the FBR to NO-releasing implants is dependent on release properties. Accordingly, in one embodiment, the present invention relates to percutaneously implanted NO-releasing glucose biosensors. In an embodiment, it is found that by extending NO-release duration, analytical merits of the sensor (i.e., accuracy, sensitivity, response time) will be maintained for extended implantation periods (>7 d). In one embodiment, the present invention relates to enhanced analytical performance of NO-releasing needle-type glucose biosensors in subjects (such as pigs) that were studied as a function of NO-release duration.

In a variation, the present invention relates to being able to solve problems associated with foreign body response and the related decrease in sensor performance for in vivo continuous glucose biosensor devices. Thus, in one embodiment, this technology will likely be useful for other sensor/electrode materials in other parts of the body (for example, those for use in the brain).

### BRIEF DESCRIPTION OF THE SEVERAL VIEWS OF THE DRAWING

FIGS. 1 A and B are a Comparison of MARD (mean absolute relative duration—described below) for (A) MAP3/NO (((3-methylaminopropyl)trimethoxysilane N-diazeniumdiolate NO donors—red circle) and control (MAP3) sensors (black, square) and (B) MPTMS-RSNO ((3-mercaptopropyl)trimethoxysilane-S-nitrosothiols—red circle) and control (MPTMS) (black, square) sensors. Significant differences (p<0.05) in the median value for the MARD are indicated with an asterisk.

FIG. 2 shows an estimation of sensor lag time via cross-correlation. MPTMS-RSNO biosensors (inverted triangle) exhibited significantly reduced lag times on days 3, 7, and 10 versus MAP3/NO sensors (circle), and MAP3 and MPTMS controls (square and triangle, respectively). Asterisks denote significant differences (p<0.05) in the median values for lag time between the MPTMS-RSNO sensors and all other sensor types.

FIG. 3A shows a comparison of sensitivity for MPTMS-RSNO ((3-mercaptopropyl)trimethoxysilane-S-nitrosothiols—red circle) and control (MPTMS) (black, square) sensors over time.

FIG. 3B shows a comparison of sensitivity for MAP3/NO (((3-methylaminopropyl)trimethoxysilane N-diazeniumdiolate NO donors—red circle) and control (MAP3) sensors (black, square) over time.

FIG. 4A shows a schematic of a NO-releasing glucose monitoring sensor.

FIG. 4B shows blood glucose concentration measurements v. time for the biosensors relative to a reference biosensor (black graph). The red circles show measurements from a reference biosensor. The good agreement shows that the biosensors of the present invention possess good accuracy.

FIG. 5 shows a biosensor with the foreign body response with a close up of the various moieties that are involved in the foreign body response.

FIGS. 6 A and B show the respective mechanisms involved in the nitric acid release. FIG. 6A shows the mechanism related to N-Dizeniumdiolate (MAP3) and FIG. 6B shows the S-nitrosothiol NO-donor mechanism (MPTMS).

FIG. 7 shows a plot of the amount of NO release over time for both the MAP3/NO (blue) and the MPTMS-RSNO (red) nanoparticles on a biosensor. Note the crossing point at 160 pmol/s cm² at about 1.5 hours.

FIG. 8A shows Representative current trace for glucose biosensor following implantation.

FIG. 8B shows distribution of estimated run-in times for NO-releasing and control sensors. Error bars indicate the total spread of data and boxes represent data points that lie in the center quartiles (25-75%).

FIG. 9 shows a Clarke error grid for MPTMS-RSNO biosensors on day 0. While daily IVGTT provided excursions into the hyperglycemic range, the majority of glucose determinations (˜70%) were made in the 50-100 mg dL−1 range. Zones labeled A and B represent clinically acceptable blood glucose measurements, while zones C, D, and E represent erroneous and progressively worse determinations.

### DETAILED DESCRIPTION OF THE INVENTION

The present invention relates to instruments and methods for in vivo analysis and the associated performance of percutaneously implanted nitric oxide (NO)-releasing amperometric glucose biosensors.

In an embodiment, the present invention relates to nitric oxide releasing glucose concentration determining biosensors that are improved relative to the biosensors that are presently available. In some embodiments the biosensors of the present invention are improved over those currently available because they are able to release nitric oxide at levels that are above those currently available. Alternatively and/or additionally, the biosensors of the present invention are improved over the biosensors that are available because the nitric oxide is released over a longer duration of time. In both instances, these improvements lead to one being able to make more precise and/or more accurate measurements, leads to greater sensitivity, allows the biosensor to detect concentrations of an analyte (for example glucose of lactate) for a longer duration, or some other advantage, or combinations thereof.

In an embodiment, the biosensors can be inserted into a subject to measure glucose concentration. Subjects that may have the biosensor inserted and/or have the biosensor used for an associated method include, but are not limited to, horses, cows, sheep, pigs, mice, dogs, cats, primates such as chimpanzees, gorillas, rhesus monkeys, and humans. In an embodiment, a subject is a human in need of having his/her glucose level measured.

Thus, in an embodiment, the present invention relates to an implantable biosensor for determining analyte concentration levels in a subject, wherein said biosensor produces and/or releases nitric oxide at the sensor-tissue interface at a level and for a duration that allows for accurate monitoring of the analyte concentration levels in said subject. In one embodiment, the nitric oxide is liberated at a level of at least about 160 pmol/s cm² for at least about 1.5 hours in phosphate buffered saline or an equivalent biological solution. The analyte may be any of a number of biological molecules that one may have an interest in monitoring. In one embodiment, the analyte may be glucose or lactate. Alternatively, other metabolites/analytes that may be monitored include cholesterol (either low density or high density lipoprotein cholesterol), oxygen, molecules related to apoptosis, molecules related to antiogenesis, steroids, or other biologically relevant molecules.

In a variation, the biosensor may produce and/or release nitric oxide at a level of at least about 300 pmol/s cm² for at least about 1.5 hours. Alternatively, the biosensor may produce and/or release nitric oxide at a level of at least about 350 pmol/s cm² for at least about 1.5 hours. Alternatively, the biosensor may produce and/or release nitric oxide at a level of at least about 400 pmol/s cm² for at least about 1.5 hours. Alternatively, the biosensor may produce and/or release nitric oxide at a level of at least about 500 pmol/s cm² for at least about 1.5 hours.

Equivalent biological solutions may be any solution that has properties similar to those of phosphate buffered saline. For example, it is contemplated and therefore within the scope of the invention that the biosensor be used in vivo. Thus, any part of a subject that is receptive to receiving a biosensor (or having one inserted) is contemplated as being part of the invention. For example, the tissue of a subject, the interstitial fluid, the skin, vasculature, subcutaneous tissue, or the blood of a subject is contemplated. Moreover, saline solutions are also contemplated as being equivalent solutions. The saline may simple be deionized water and a physiologically relevant amount of sodium chloride. Alternatively, the saline solution may also contain biologically relevant sugars such as dextrose, glucose, allose, gulose, galactose, sucrose, malose, etc. Other equivalent solutions include those buffers that are used in biological systems (generally they have a pH that is a biologically relevant range). This include TRIS (tris(hydroxymethyl)aminomethane) buffer, TAPS (3-[[1,3-dihydroxy-2-(hydroxymethyl)propan-2-yl]amino]propane-1-sulfonic acid) buffer, bicine (2-(Bis(2-hydroxyethyl)amino)acetic acid) buffer, Tricine (N-(2-Hydroxy-1,1-bis(hydroxymethyl)ethyl)glycine) buffer, TAPSO (3-[[1,3-dihydroxy-2-(hydroxymethyl)propan-2-yl]amino]-2-hydroxypropane-1-sulfonic acid) buffer, HEPES (2-[4-(2-hydroxyethyl)piperazin-1-yl]ethanesulfonic acid) buffer, TES (2-[[1,3-dihydroxy-2-(hydroxymethyl)propan-2-yl]amino]ethanesulfonic acid) buffer, MOPS (3-morpholinopropane-1-sulfonic acid) buffer, PIPES (1,4-Piperazinediethanesulfonic acid) buffer, cacodylate (Dimethylarsinic acid) buffer, SSC (saline sodium citrate) buffer, MES (2-(N-morpholino)ethanesulfonic acid) buffer, and succinic acid (2(R)-2-(methylamino)succinic acid) buffer.

In an embodiment, the present invention relates to a biosensor for determining glucose levels in a subject, wherein said biosensor comprises a coating that is doped with one or more macromolecular NO-donor scaffolds as a method to produce nitric oxide at the sensor-tissue interface. In one variation, the one or more macromolecular NO-donor scaffolds comprise MAP3 or MPTMS nanoparticles, or a combination of the two. In one embodiment, other NO producing and/or releasing macromolecules are contemplated and are therefore within the scope of the invention. In one variation, the dopant concentration is sufficient so as to produce the requisite response. In one variation, if MAP3 and MPTMS nanoparticles are used, the dopant concentration may be at least about 72 and 48 mg mL⁻¹ for the MAP3 and the MPTMS nanoparticles, respectively. Alternatively, a slightly lower concentration may be used. In one embodiment, the dopant concentration is determined so as to give sufficient NO production over a given duration. In one embodiment, the nitric oxide may be produced at a level of at least about 160 pmol/s cm² for at least about 1.5 hours in phosphate buffered saline or an equivalent biological solution.

In one embodiment, the biosensors of the present invention are superior to those that are currently available because they can measure analyte (for example, glucose or lactate) concentration for longer durations. Thus, in one embodiment, the biosensor of the present invention is able to accurately determine glucose levels using the biosensor in a subject at least about 3 days after insertion of the biosensor in the subject. Alternatively, the biosensor may be able to accurately determine glucose levels using the biosensor in the subject at least about 4 days, or alternatively, five days, or alternatively, six days, or alternatively seven days, or alternatively, eight days, or alternatively, nine days, or alternatively, ten days after insertion of the biosensor in the subject. It is contemplated and therefore within the scope of the invention that the biosensor may work (i.e., give accurate measurements) for more than 7 days, or alternatively, 10 days after insertion of the biosensor into a subject.

In one embodiment, the accuracy of the biosensor can be compared to instruments that use the finger prick method. Thus, in one variation, when the term “accurately” is used, it is meant relative to a method and/or instruments that use the finger prick method (a hand held glucometer). Moreover, in one variation, the accuracy of the instrument is such that the difference between the biosensor and a method/instrument using the pin prick method is no more than about a 25% difference, or alternatively, no more than about a 20% difference, or no more than about a 15% difference, or more than about a 10% difference, or alternatively, no more than about a 5% difference.

In one variation, the biosensors that give accurate measurements for long duration may comprise MAP3 or MPTMS nanoparticles, or a combination of the two.

In one embodiment, the biosensor(s) that produce(s) and/or release(s) nitric oxide has a maximal amount of nitric oxide that is released. In one variation, the maximal level is not more than about 700 pmol/s cm², or alternatively, not more than about 650 pmol/s cm², or alternatively, not more than about 600 pmol/s cm², or alternatively, not more than about 550 pmol/s cm², or alternatively, not more than about 500 pmol/s cm², or alternatively, not more than about 450 pmol/s cm², or alternatively, not more than about 400 pmol/s cm², or alternatively, not more than about 300 pmol/s cm². In these embodiments, when levels above these levels are produced and/or released, the performance of the biosensor(s) tend(s) to suffer.

The term “about,” as used herein, when referring to a value or to an amount of mass, weight, time, volume, or percentage is meant to encompass variations of .+−0.20% or .+−. 10%, or .+−.5%, or +−.1%, or +−.0.1% from the specified amount, as such variations are appropriate to perform the disclosed method(s).

In one embodiment, the present invention relates to a method of determining glucose concentration levels in a subject by insertion of a biosensor in said subject, the biosensor comprising a polyurethane coating that is doped with one or more of MAP3 or MPTMS nanoparticles designed to release nitric oxide, wherein the biosensor has been calibrated in a buffer to release a nitric oxide level of at least about 160 pmol/s cm² for at least about 1.5 hours.

Alternatively, the method contemplates being able to determine glucose levels using the biosensor in the subject at least about 4 days, or alternatively, five days, or alternatively, six days, or alternatively seven days, or alternatively, eight days, or alternatively, nine days, or alternatively, ten days after insertion of the biosensor in the subject. It is contemplated and therefore within the scope of the invention that the method may have a biosensor that may work (i.e., give accurate measurements) for more than 10 days after insertion of the biosensor into a subject.

In an embodiment, the amount of nitric oxide that is produced and/or released is sufficient to produce and/or release an effective amount for performing an accurate measurement. The term “effective amount” is used herein to refer to an amount of the therapeutic composition (e.g., a composition comprising a nitric oxide-releasing particle) sufficient to produce a measurable biological response, such as an amount being able to accurately measure an analyte. Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including the activity of the composition, formulation, the route of administration, combination with other drugs or treatments, severity of the condition being treated, and the physical condition and prior medical history of the subject. In one variation, a minimal dose is administered, and dose is escalated in the absence of dose-limiting toxicity to a minimally effective amount.

In an embodiment, the biosensor may have a polyurethane coating and nanomolecules that release nitric oxide. In one variation, the nanomolecules may comprise MPTMS ((3-mercaptopropyl)trimethoxysilane) or MAP3 ((3-methylaminopropyl)trimethoxysilane). These nanomolecules may have a moiety associated with them that are designed to release nitric oxide. These may include nitrosated thiol-containing nanoparticles or molecules that have undergone N-diazeniumdiolation of a secondary amine in the nanoparticle. Other nitric oxide generating and/or releasing moieties are contemplated and therefore within the scope of the invention. One such method involves organodiselenides (e.g., selenocystamine (SeCA) and 3,3-diselenodipropionic acid (SeDPA)), and certain selenium containing enzymes (e.g., glutathione peroxidase (GPx)), and organoditellurides (e.g., 5,5-ditelluro-2,2-dithiophenecarboxlyic acid (DTDTCA)), which can carry out catalytic NO generation chemistry by decomposing endogenous RSNO compounds. Alternatively, nitric oxide can be produced from the electrochemical reduction of nitrite using a copper(II)-tri(2-pyridylmethyl)amine (Cu(II)TPMA) complex as a mediator.

In some embodiments, the NO donor is selected from the group consisting of a diazeniumdiolate, a nitrosamine, a hydroxylamine, a nitrosothiol, a hydroxylamine, and a hydroxyurea. In some embodiments the NO donor is covalently bound to one of the interior region, the exterior region, the core, or to combinations thereof. In some embodiments the NO donor is encapsulated in one of the interior region, the exterior region, the core, or to combinations thereof. In some embodiments the NO donor is associated with part of the particle via a non-covalent interaction selected from the group consisting of Van der Waals interactions, electrostatic forces, hydrogen bonding, or combinations thereof.

In some embodiments, the NO-releasing particles can be incorporated into polymeric films. Such incorporation can be through physically embedding the particles into polymer surfaces, via electrostatic association of particles onto polymeric surfaces, or by covalent attachment of particles onto reactive groups on the surface of a polymer. Alternatively, the particles can be mixed into a solution of liquid polymer precursor, becoming entrapped in the polymer matrix when the polymer is cured. Polymerizable groups can also be used to functionalize the exterior of the particles, whereupon, the particles can be co-polymerized into a polymer during the polymerization process. Suitable polymers into which the NO-releasing particles can be incorporated include polyolefins, such as polystyrene, polypropylene, polyethylene, polytetrafluoroethylene, and polyvinylidene, as well as polyesters, polyethers, polyurethanes, and the like. In particular, polyurethanes can include medically segmented polyurethanes. A generalized structure for a medically segmented polyurethane can include hard segments, e.g., moieties that are relatively rigid, and soft segments, e.g., moieties having more degrees of freedom that can exist in a number of alternate, inter-converting conformations. Medically segmented polyurethanes can also include one or more expander moieties, such as alkylene chains, that add additional length or weight to the polymer. Such polyurethanes are also generally non-toxic.

In an embodiment, the NO-releasing particles can be incorporated into detergents, such as, but not limited to, anti-microbial soaps. For example, NO-release in particles embedded in bar soaps can be triggered by contact with water and/or a drop in pH upon use. As the outer surface of the bar is eroded or dissolved, additional particles within the bar surface become exposed for subsequent uses of the bar. NO-releasing particles also can be suspended in liquid soaps. Such soaps or detergents can be used for personal hygiene or to provide anti-microbial treatments for fibers. Such soaps or detergents can also be used to treat household surfaces or any surface in a hospital or other medical environment that may be exposed to microbes such as bacteria, fungi or viruses.

### Experimental Section

### Materials

Glucose oxidase (GOx; type VII from Aspergills niger, >100,000 units g⁻¹), D-(+)-glucose anhydrous, acetaminophen (AP), L-ascorbic acid (AA), urea (UA), phenol, and sodium methoxide (5.4 M in methanol) were purchased from Sigma (St. Louis, Mo.). Tetrahydrofuran (THF), ethanol (EtOH), aqueous ammonium hydroxide (30 wt %), and all salts were purchased from Fisher Scientific (St. Louis, Mo.) Tetraethyl orthosilicate (TEOS), (3-mercaptopropyl)trimethoxysilane (MPTMS), and (3-methylaminopropyl)trimethoxysilane (MAP3) were purchased from Gelest (Tullytown, Pa.). Methyltrimethoxysilane (MTMOS) was purchased from Fluka (Buchs, Switzerland). Cetyltrimethylammonium bromide (CTAB) was purchased from Acros Organics (Geel, Belgium). Hydrothane (AL25-80A) polyurethane (HPU) was a gift from AdvanSource Biomaterials (Wilmington, Mass.). Tecoflex (SG-85A) polyurethane (TPU) was a gift from Lubrizol (Cleveland, Ohio). Steel wire (356 μm dia.) was purchased from McMaster-Carr (Atlanta, Ga.). Argon, nitrogen, oxygen, and nitric oxide calibration gas (25.87 ppm in nitrogen) were purchased from Airgas National Welders (Raleigh, N.C.). Nitric oxide gas was purchased from Praxair (Danbury, Conn.). Water was purified using a Millipore Milli-Q UV gradient A10 system (Bedford, Mass.) to a resistivity of 18.2 MΩ·cm and a total organic content of ≦6 ppb. All other chemicals were reagent grade and used as received.

### Synthesis of NO-Releasing Silica Nanoparticles

Synthesis of NO-releasing silica nanoparticles was carried out as described previously. Briefly, MPTMS particles were synthesized via the co-condensation of MPTMS (70 mol %) and TEOS. The thiol-containing nanoparticles were nitrosated by reaction with acidified nitrite in the dark at 0° C. for 2 h. Mesoporous MAP3 silica nanoparticles were prepared via a surfactant-templated co-condensation of TEOS in the presence of CTAB, followed by removal of CTAB and surface-grafting of MAP3 to the particle surface. Subsequent N-diazeniumdiolation of the secondary amine-containing nanoparticles was carried out under high pressures of NO (10 atm) at room temperature for 3 d in the presence of sodium methoxide. The sizes and total NO-release payloads of the silica nanoparticles are shown in Table S2 below.

TABLE S2
Nanoparticle NO Donor Characterization

| Nanoparticle NO Donor | [NO]_{T} (μmol mg⁻¹) | Particle Diameter |
|---|---|---|
| MAP3 | 2.03 ± 0.20 | 820 ± 70 |
| MPTMS | 3.36 ± 0.62 | 620 ± 80 |

^{a}Nanoparticle diameter estimated via scanning electron microscopy

### Preparation of NO-Releasing Mock Sensors

Steel wire was cut in 7 cm pieces and cleaned by sonication in EtOH for 10 min. Polymer solutions containing the macromolecular NO-release scaffolds were prepared by dispersing MAP3 or MPTMS particles (72 and 48 mg mL⁻¹, respectively) in an 80 mg mL⁻¹ solution of 50:50 wt % HPU/TPU in 1:1 EtOH:THF. Wire substrates were modified by dip coating (5 mm s⁻¹ with a 5-s hold time) four times into the particle-containing PU solution using a DipMaster™ 50 dip coater (Chemat Technology, Inc.; Northridge, Calif.) with 30 min drying periods under ambient conditions between dips. A final TPU topcoat was applied by dip coating into a 40 mg mL⁻¹ TPU solution in THF.

### Characterization of NO-Releasing Wire Substrates

Nitric oxide release from steel wire substrates was measured in real time using a Sievers 280i chemiluminescence NO analyzer (NOA; Boulder, Colo.). Generation of NO from PU films was detected indirectly by the formation of a chemiluminescent product (NO₂*) upon reaction of NO with ozone. The NOA was calibrated using an atmospheric gas sample passed through a Sievers NO zero filter (0 ppb) and 25.9 ppm NO in N₂. Substrates were immersed in deoxygenated phosphate buffered saline (PBS; 0.01 M, pH 7.4) at 37° C. The liberated NO from PU films was carried to the NOA by a stream of N₂, bubbled into solution at a volumetric flow rate of 75 mL min⁻¹. For films containing S-nitrosothiol NO-donors (e.g., MPTMS particles), the sample flask was shielded from light and 500 μM DTPA was added to the PBS buffer to chelate trace copper. Data output from the NOA was collected every 1 s, allowing for near real-time monitoring of NO generated from the films.

The stability of silica particles in PU films was assessed using inductively coupled plasma optical emission spectrometry (ICP-OES). Modified wire substrates were immersed in PBS buffer and incubated at 37° C. for 10 d. The degree of particle leaching into soak solutions was determined by monitoring the silicon emission intensity at 251.611 nm using a Prodigy high dispersion ICP (Teledyne Leeman Labs; Hudson, N.H.).

### Fabrication and Benchtop Performance of NO-Releasing Needle-Type Glucose Sensors

Bare needle-type glucose sensors (Pinnacle Technology, Inc., Lawrence, Kans.), composed of an integrated silver/silver chloride (Ag/AgCl) pseudo-reference electrode wound around a 90:10 platinum/iridium (Pt/Ir) working electrode (127 μm dia., ˜1 mm length), were functionalized by the successive deposition of a polyphenol selectivity layer, a GOx enzyme layer, a NO-releasing flux-limiting membrane, and a polyurethane topcoat. Following deposition of the selectivity and enzyme layers, sensors were coated with a PU diffusion-limiting/NO-releasing layer by dip-coating into a particle-containing PU solution. A TPU topcoat was then applied as an additional layer. Control sensors were coated using PU solutions containing MAP3 or MPTMS nanoparticles (72 and 48 mg mL⁻¹, respectively) that were not functionalized with N-diazeniumdiolate or S-nitrosothiol NO donors.

### In Vivo Protocol for Assessing Biosensor Analytical Performance

The animal protocol used in this study was IACUC approved. The in vivo performance of glucose biosensors was evaluated in Yorkshire-type piglets (n=10) weighing approximately 7-15 kg. Details regarding sensor implantation and operation are provided in the Supporting Information. Biosensor performance was evaluated on 0, 1, 3, 7, and 10 d after sensor implantation. A peripherally-inserted central catheter was placed in an external jugular vein for blood draws. Reference blood glucose (BG) concentrations were measured every 10 min for 6-8 h using a One Touch® Ultra glucometer (LifeScan, Inc.; Milpitas, Calif.) for comparison to sensor data. During glucose sensor evaluation, pigs were fasted and sedated with propofol (2 mg kg⁻¹h⁻¹) administered through a catheter in a peripheral ear vein. Once on the day of implantation and three times daily thereafter, the swine were challenged with an intravascular glucose tolerance test (IVGTT; 0.7 g kg⁻¹, 50 wt/% dextrose, 1-1.5 h duration), administered over 30 s through the peripheral catheter, to assess the ability of glucose sensors to track changing blood glucose concentrations. On day 10, pigs were euthanized and the sensors were explanted by removal of the surrounding tissue en bloc. Post-explanation, sensors were imaged using environmental scanning electron microscopy (ESEM; FEI Quanta 200 Field Emission Gun; Hillsboro, Oreg.).

### Data Analysis

Sensor current traces were filtered and analyzed using custom MATLAB scripts (Mathworks, Inc.; Natick, Mass.). A finite impulse response (FIR) filter was used to attenuate large noise spikes caused by pig motion and potentiostat RF transmitter dropout. A one-minute median filter was used to further smooth the data before pairing sensor current traces with reference measurements. Glucose sensors were calibrated with respect to reference BG measurements once per day using a two-point retrospective calibration. One point for calibration was taken at a stable glucose baseline (i.e., prior to the first IVGTT), while the second point was taken at a stable point after the first dextrose administration with at least a 15 mg dL⁻¹ difference between BG concentrations. The slope of a linear trend line connecting these two points was taken as the apparent in vivo biosensor sensitivity on each day, expressed as mean values±standard deviation. The method of Poincaré was used to approximate the time delay at which the correlation between the reference and calibrated sensor signals was greatest, using R² as the agreement criterion. This delay was determined at ˜5 min and used to correct sensor data on each day for the physiological time lag that characterizes mass transfer of glucose from blood to tissue. After sensor implantation, the “run-in” time (i.e., the time required for sensors to achieve a stable background current) was estimated by determining the period over which two consecutive sensor measurements agreed with their respective reference measurements within 20%.

Sensor performance was determined using numerical and clinical accuracy metrics. The mean absolute relative deviation (MARD) for a data set collected by a single sensor (˜25-35 measurements) was used to characterize sensor numerical accuracy at each time point. Sensor MARD was calculated using equation 1 below, where CGM and BG are the blood glucose values determined by the sensor and reference glucometer, respectively.

$$MARD = Mean ( \frac{\vert CGM - BG \vert}{BG} * 100 ) \qquad ( 1 )$$

Additionally, the International Standards Organization (ISO) criteria for glucose monitor performance was used to assess sensor numerical accuracy by separately calculating the percentage of glucose measurements determined by sensors that were within (1)±15 mg dL⁻¹ of the paired reference determination when BG was ≦70 mg dL⁻¹ and (2)±20% of the paired reference determination when BG was >70 mg dL⁻¹. Sensor clinical accuracy was determined using Clarke error grid analysis (EGA) by quantifying the percentage of blood glucose determinations falling in zones A and B of the error grid. Cross-correlation of the reference signals and raw sensor current traces was used to estimate sensor lag time, with possible lag times restricted to >100 s. Values for MARD and lag time are expressed as mean values±standard error of the mean. Differences in median values for sensor MARD, lag time, and sensitivity between NO-releasing and control sensors were analyzed using a two-tailed non-parametric Mann-Whitney U test.

### Results and Discussion

Nitric oxide-releasing polyurethanes were selected as sensor coatings for evaluating the effect of NO-release duration on in vivo glucose biosensor performance. Total NO payloads sufficient for minimizing inflammation (i.e., >1 μmol cm⁻²) with varied NO-release durations (<1 h to >14 d) were achieved by tuning the PU properties (i.e., water uptake) and NO donor type. IT should be noted that sensor response is not negatively affected by NO release from PU coatings at a working electrode potential of +600 mV vs. Ag/AgCl. The versatile NO-release kinetics and compatibility with amperometric glucose sensing make NO-releasing polyurethanes an ideal platform for assessing the effects of NO release on in vivo glucose biosensor performance.

### In Vitro Characterization of NO-Releasing Glucose Sensors

Wire substrates, selected to mimic the geometry and size of a needle-type glucose sensor, were modified with NO-releasing PU coatings via a dip-coating procedure. A hydrophobic TPU topcoat was employed to both minimize any leaching of the macromolecular NO donors and eliminate the surface roughness introduced by nanoparticle dopants. Undoubtedly, the physical properties (i.e., roughness) of an implant surface will affect the FBR. The stability of the nanoparticle-doped PU coatings in PBS was investigated over 10 d by analyzing the silicon content of soak solutions using ICP-OES. While silica is intrinsically biocompatible and considered non-toxic, the resulting changes in coating structure or potential tissue inflammation may affect the performance of glucose sensors in vivo. For coatings doped with NO-releasing MPTMS-RSNO particles as well as controls, leaching of silica particles from the PU matrix was undetectable (<2%). Slight leaching (10.8±2.9% of the total incorporated silica) was observed from coatings containing NO-releasing MAP3/NO particles. Interestingly, the majority of the observed leaching occurred during the first 4 h, suggesting some instability associated with encapsulating the charged N-diazeniumdiolate NO donor moieties within the polyurethane coating (data not shown).

The effect of NO-release duration on in vivo sensor performance was studied using two different macromolecular NO release systems: N-diazeniumdiolate NO donors and S-nitrosothiol modified silica nanoparticles (MAP3/NO and MPTMS-RSNO, respectively). Briefly, N-diazeniumdiolate NO donors undergo proton-initiated decomposition in aqueous milieu to generate NO. Conversely, NO-release from S-nitrosothiols may be triggered using light or Cu(I), but also decompose sluggishly through thermal mechanisms in vivo. To simulate in vivo conditions, NO release from PU films was measured in PBS at 37° C. For MPTMS-RSNO coatings, thermal decomposition of the S-nitrosothiol moieties was achieved using a light-shielded sample flask and the addition of DTPA to chelate trace copper. By appropriate selection of the nanoparticle dopant concentration (72 and 48 mg mL⁻¹ for MAP3/NO and MPTMS-RSNO particles, respectively) the inventors attained similar total NO payloads (˜3.1 μmol cm⁻²) for both coating formulations (Table 1). Of note, NO payloads from these coatings were more than two times greater than the xerogel coatings utilized by Hetrick et al. (˜1.35 μmol cm⁻²) and similar in magnitude to those employed by Nichols et al. (2.7-9.3 μmol cm⁻²)-both of which proved effective at reducing the FBR to subcutaneous implants.

TABLE 1
Nitric oxide release from polyurethane coatings doped
with NO-releasing MPTMS-RSNO and MAP3/NO nanoparticles.

| NO-Release Merits | MPTMS-RSNO | MAP3/NO |
|---|---|---|
| [NO]ₘₐₓ (pmol cm⁻² s⁻¹) | 551.4 ± 130.0 | 685.8 ± 11.4 |
| tₘₐₓ (min)^{a} | 1.68 ± 0.20 | 23.80 ± 7.17 |
| t_{1/2} (h)^{b} | 6.29 ± 2.07 | 0.93 ± 0.17 |
| [NO]_{8 h} (pmol cm⁻² s⁻¹) | 14.0 ± 3.9 | 13.0 ± 3.2 |
| [NO]_{12 h} (pmol cm⁻² s⁻¹) | 9.8 ± 3.8 | 3.7 ± 1.5 |
| [NO]_{24 h} (pmol cm⁻² s⁻¹) | 3.3 ± 0.2 | 0^{c} |
| [NO]_{48 h} (pmol cm⁻² s⁻¹) | 1.0 ± 0.1 | 0^{c} |
| [NO]_{72 h} (pmol cm⁻² s⁻¹) | 0.5 ± 0.0 | 0^{c} |
| [NO]_{168 h} (pmol cm⁻² s⁻¹) | 0.5 ± 0.0 | 0^{c} |
| [NO]_{T} (μmol cm⁻²)^{d} | 3.14 ± 0.26^{e} | 3.11 ± 0.27 |
| t_{d} (h)^{f} | 74.6 ± 16.6 | 16.0 ± 4.4 |

^{a}Time required to reach maximum NO flux.
^{b}Half-life for NO-release from PU films.
^{c}Nitric oxide release was below the limit of detection of the NOA.
^{d}Total amount of NO released.
^{e}Measured by irradiation of the sample flask with 200 W light.
^{f}Determined at the time at which 99% of the total NO was released.

Upon immersion in PBS, MAP3/NO films exhibited a large initial NO flux ([NO]ₘₐₓ=685.8±11.4 pmol cm⁻²s⁻¹) and released 99% of their total NO payload within ˜16 h, with no additional NO release measurable beyond 24 h. Such duration (16 h) was similar to that reported to improve glucose sensor accuracy by Gifford and coworkers (12-18 h). Similarly, MPTMS-RSNO films showed a large initial NO flux ([NO]ₘₐₓ=551.4±130.0 pmol cm⁻²s⁻¹), with a rapid decrease to ˜14.0 pmol cm⁻² s⁻¹ at 14 h. In contrast to the MAP3/NO films, MPTMS-RSNO coatings required ˜3.1 d to release 99% of their total NO payload, with NO release (0.5 pmol cm⁻²s⁻¹) still measurable at ˜7 d. Even such low levels of NO are physiologically relevant, as vascular endothelial cells release NO at 1-7 pmol cm⁻²s⁻¹ to prevent platelet activation. Additionally, similar NO fluxes (1.5-30 pmol cm⁻² s⁻¹) inhibit in vitro bacterial adhesion to surfaces.

Nitric oxide release from the outer glucose sensor membrane did not impact biosensor response. After an initial hydration period of 3-4 h, the glucose sensitivities of NO-releasing and control sensors were comparable and remained constant (1.3-2.3 nA mM⁻¹) over 10 d in PBS at 37° C. for all membrane formulations. In the absence of pre-conditioning, sensors exhibited poorer dynamic range and longer response times to changes in glucose concentration during the first several hours of testing (data not shown). Both NO-releasing and control sensors exhibited acceptable response times (<40 s) to an increase in glucose concentration of 5.6 mM. All sensors responded linearly to glucose between 1-12 mM after pre-conditioning in PBS. Furthermore, the amperometric selectivity coefficients for glucose over acetaminophen, ascorbic acid, and urea were 0.82±0.30, 0.49±0.11, and 0.03±0.01, respectively for blank sensors (i.e., sensors that were coated solely with polyurethane). Selectivity for glucose was sufficient.

### In Vivo Biosensor Run-in Time, Glucose Sensitivity, and Clarke Error Grid

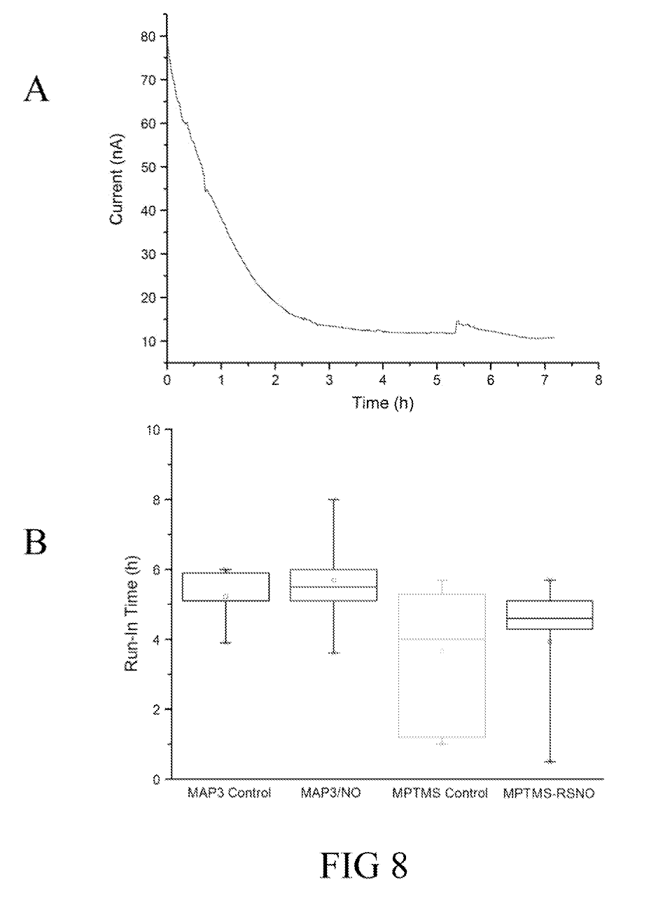

Following implantation, both NO-releasing and control biosensors displayed a run-in period (i.e., the time required to achieve a stable baseline current) during which the sensor response was erratic (FIG. 8). While one might expect a reduced run-in time for NO-releasing sensors versus control sensors in rodents, the present studies observed no significant differences in run-in time between NO-releasing sensors and controls, with all four sensor configurations requiring ˜3-6 h to achieve a steady background current. The source of this discrepancy is unclear, but a number of variables (e.g., different animal model, implant method, and extended sensor hydration time) may have contributed to this result.

The potential analytical performance benefits of NO-releasing amperometric glucose biosensors were evaluated in a healthy swine model. The use of digital noise filters was used to achieve stable current traces due to swine motion and intermittent potentiostat RF transmitter dropout. The filtering algorithms were restricted to those compatible with real-time continuous glucose monitoring. The FIR and median filters sufficiently improved signal quality without introducing an undesirable artificial time delay (>20%) between sensor and reference signals. Subsequently, sensors were calibrated by comparison to corresponding reference blood glucose measurements using a two-point retrospective calibration. While a one-point calibration (which assumes a negligible background current) has been suggested to be superior to the two-point calibration, the in vivo background in this study was substantial (6-10 nA) compared to the in vitro baseline (1-3 nA). Accordingly, a two-point calibration was used. Other researchers have also reported disparities between in vitro and in vivo sensor baseline currents. Despite minimizing the artificial delay caused by filtering, a physiological lag between the sensor signal and reference BG measurements was still observed. This delay arises from the slow mass transfer of glucose from the vasculature to the tissue and ultimately the sensor. An analysis of sensor performance on day 0 via the method of Poincaré indicated a ˜5-min lag between the reference signal and calibrated sensor signal. This lag time was thus accounted for in all remaining data sets (days 1, 3, 7, and 10) by shifting the reference signal in time relative to the sensor signal.

The clinical accuracies of NO-releasing and control in vivo glucose biosensors were first assessed via the Clarke error grid. The percentage of BG measurements falling in zones A and B (clinically accurate and clinically benign determinations, respectively) of the error grid are shown in Table 2. On the day of implantation (day 0), the MAP3/NO-based sensors performed slightly worse than control sensors, with a 2% difference in the percentage of determinations in zones A and B. However, the performance of MAP3/NO sensors on days 1 and 3 was superior to controls, with >7% difference in the percentage of clinically accurate and acceptable determinations. Concomitant with improved clinical performance, sensors that rapidly released NO were characterized as having greater glucose sensitivity on days 1 and 3 (0.59±0.54 and 0.59±0.40 nA mM⁻¹, respectively) versus controls (0.14±0.09 and 0.18±0.04 nA mM⁻¹, respectively). However, the MAP3/NO sensors exhibited similar clinical accuracy and glucose sensitivity to control sensors at implant periods beyond three days (e.g., days 7 and 10), suggesting that sensor performance is improved during periods of active NO release. The trends in sensor clinical performance and glucose sensitivity correlate well with the NO-release kinetics from the sensors, with clear benefits to sensor performance early during in vivo use (i.e., days 1 and 3) but no improvements after the NO supply was exhausted. Of note, others have noted no decrease in the FBR (>1 w) for implants with rapid NO-release, suggesting that inflammation may be the primary culprit for decreased sensor performance beyond 3 d. Unexpectedly, the MPTMS-RSNO based sensors exhibited similar clinical accuracy to MPTMS control sensors throughout the 10 d in vivo study. The sensitivity of the MPTMS-RSNO sensors to glucose appeared slightly greater than MPTMS controls beyond day 0, but these differences were not significant (p>0.05). This result may be due to the low, sustained NO fluxes released from sensors when compared to the MAP3/NO-based sensors (Table 1).

TABLE 2
Clinical performance and apparent in vivo sensitivity of glucose biosensors.

| | | MAP3 | | MPTMS | MPTMS- |
|---|---|---|---|---|---|
| Day | | Control | MAP3/NO | Control | RSNO |
| 0 | % Points in Zones | 89.6 | 87.6 | 91.0 | 94.7 |
| | N^{a} | 183 | 105 | 311 | 321 |
| | Sensitivity (nA mM⁻ | 0.90 ± 0.87 | 0.72 ± 0.40 | 0.74 ± 0.47 | 0.60 ± 0.30 |
| 1 | % Points in Zones | 78.6 | 86.2 | 90.6 | 89.1 |
| | N^{a} | 168 | 174 | 224 | 347 |
| | Sensitivity (nA mM⁻ | 0.14 ± 0.09 | 0.59 ± 0.54^{b} | 0.29 ± 0.18 | 0.39 ± 0.17 |
| 3 | % Points in Zones | 84.8 | 92.0 | 81.7 | 83.9 |
| | N^{a} | 169 | 173 | 180 | 124 |
| | Sensitivity (nA mM⁻ | 0.18 ± 0.04 | 0.59 ± 0.40^{b} | 0.24 ± 0.16 | 0.49 ± 0.18 |
| 7 | % Points in Zones | 93.2 | 94.2 | 88.3 | 88.1 |
| | N^{a} | 115 | 87 | 157 | 69 |
| | Sensitivity (nA mM⁻ | 0.23 ± 0.15 | 0.39 ± 0.26 | 0.20 ± 0.07 | 0.45 ± 0.19 |
| 10 | % Points in Zones | 84.8 | 81.4 | 91.8 | 84.9 |
| | N^{a} | 138 | 97 | 135 | 66 |
| | Sensitivity (nA mM⁻ | 0.16 ± 0.06 | 0.20 ± 0.13 | 0.09 ± 0.02 | 1.3 ± 1.1 |

^{a}Total number of measurements.
^{b}Significantly different at p < 0.05.

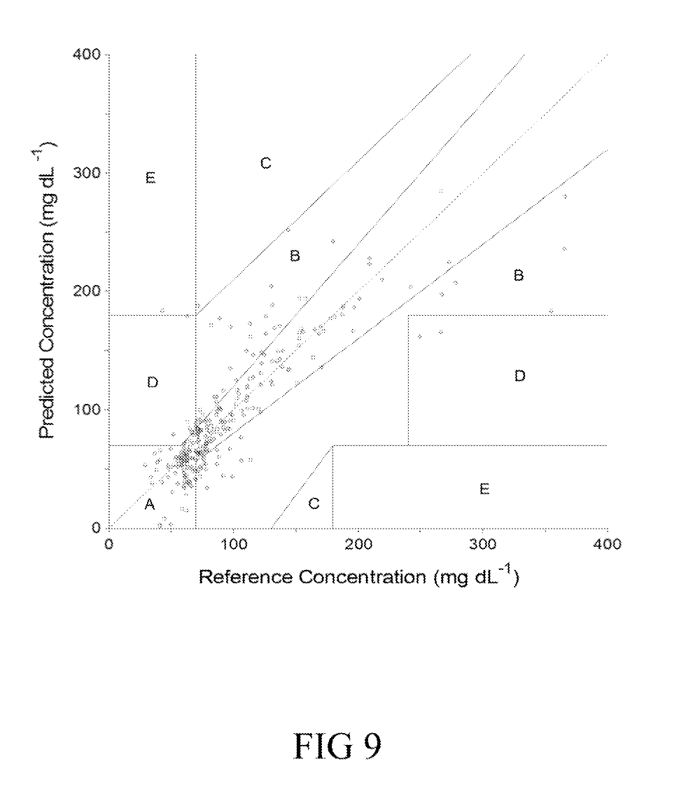

Of importance, the majority (˜70%) of BG determinations were obtained in the 50-100 mg dL⁻¹ range, as shown in a representative Clarke error grid analysis (see FIG. 9). In addition to the similarities between swine and humans (e.g., skin, vasculature, subcutaneous tissue composition) which render the pig an appropriate model for evaluating in vive biosensors, baseline blood glucose concentrations obtained in this study were comparable to human euglycemic levels. As maintenance of euglycemia increases the propensity of diabetic individuals to enter the hypoglycemic BG range, the Clarke error grid presents austere requirements for sensor accuracy in this region. Thus, the error grid analysis presented herein is at BG levels clinically and physiologically pertinent to humans.

### Biosensor Numerical Accuracy and Adherence to ISO Criteria

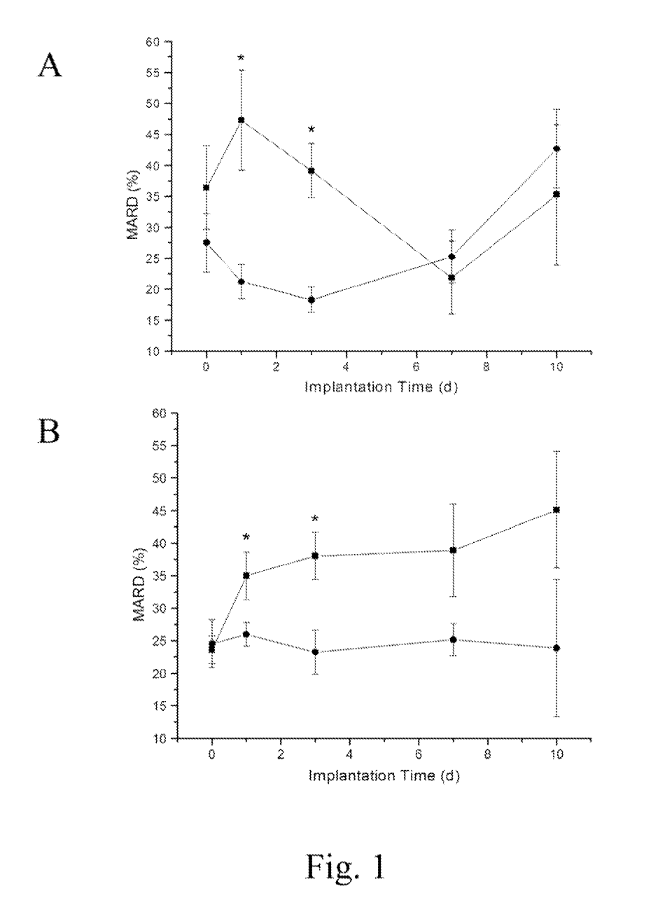

To evaluate in vivo biosensor performance in more detail, the sensor numerical accuracy was represented using the MARD of each sensor from corresponding reference values. While the Clarke error grid measures sensor accuracy based on the clinical implications of a given BG measurement, the MARD represents a statistical entity that exemplifies the average percent deviation of the sensor from a reference. Additionally, ISO criteria for in vivo glucose biosensor performance was considered as a metric for numerical accuracy because it can be used to assess sensor accuracy in both hypoglycemic (≦70 mg dL⁻¹) and euglycemic/hyperglycemic (>70 mg dL⁻¹) BG ranges separately. A comparison of the numerical accuracies for control and NO-releasing sensors is shown in FIG. 1. The analytical performance of MAP3/NO-based sensors on days 1 and 3 was superior to MAP3 (control) sensors. The improvements in numerical accuracy agree with the increased clinical accuracy and greater glucose sensitivity for the more rapid NO-releasing sensors. Furthermore, the performance of the MAP3/NO-based sensors was observed to worsen beyond 3 d implantation. The desirably lower MARD for rapid NO-releasing glucose sensors is attributed to the improved accuracy in both the hypoglycemic and euglycemic/hyperglycemic ranges, as shown in Table 3. Indeed, >55% of the total BG determinations obtained by MAP3/NO-based sensors agreed well with corresponding reference measurements in both BG ranges on days 1 and 3. Unexpectedly, the MARD for control (MAP3) sensors was lowest at 7 d implantation (21.9±13.1%). Despite the inconsistent numerical accuracy for control sensors, the analytical performance was comparable to NO-releasing sensors at both 7 and 10 d.

Although the clinical accuracy of the MPTMS-RSNO based sensors was comparable to controls, the numerical accuracy of NO-releasing sensors remained constant (MARD range 22.2-26.0%) throughout the experiment. Furthermore, the sensors that released NO for extended durations exhibited a significantly lower MARD on days 1 and 3 (26.0±5.1 and 23.9±8.6%, respectively) versus controls (34.3±10.9 and 38.8±10.4%, respectively). This can be attributed to the good agreement between MPTMS-RSNO sensors and reference measurements to the increased accuracy of the NO-releasing biosensors in both the hypoglycemic and euglycemic/hyperglycemic BG ranges. The percentage of determinations for MPTMS-RSNO based sensors that adhered to ISO criteria was typically >50% throughout implantation, while control sensor performance worsened with implant duration, particularly in the hypoglycemic range. The stable biosensor response provided by the sustained NO-releasing sensor membranes highlights the utility of having more extended NO release for continuous glucose monitoring.

TABLE 3
ISO criteria for NO-releasing and control sensors.

| | MAP3 | | MPTMS | MPTMS- |
|---|---|---|---|---|
| | Control | MAP3/NO | Control | RSNO |
| Day | (%) | (%) | (%) | (%) |
| 0 | 58.0^{a}/50.0^{b} | 51.9/61.5 | 55.7/60.0 | 60.2/67.0 |
| 1 | 37.9/39.2 | 55.6/56.7 | 45.9/59.7 | 55.5/59.4 |
| 3 | 52.9/47.7 | 65.6/57.3 | 39.5/57.3 | 58.5/74.7 |
| 7 | 62.5/62.7 | 42.1/57.8 | 35.5/45.2 | 42.1/52.0 |
| 10 | 55.6/54.9 | 30.6/45.9 | 15.0/34.8 | 63.6/45.5 |

^{a}Calculated as the percentage of determinations within 15 mg dL⁻¹ of the reference measurement when BG ≦ 70 mg dL⁻¹
^{b}Calculated as the percentage of determinations within 20% of the reference measurement when BG > 70 mg dL⁻¹

Of importance, the NO-release kinetics also correlated with the magnitude of the improvement in numerical accuracy for NO-releasing sensors versus controls. For example, MAP3/NO-based sensors showed vastly decreased MARD versus MAP3 (control) sensors on day 1 (22.0±6.6 and 47.3±8.1%, respectively), whereas sensors with longer NO-release durations (MPTMS-RSNO) exhibited more modest improvements relative to controls (28.4±5.9 and 34.3±10.9%, respectively). However, the differences in the MARD between MAP3/NO and MPTMS-RSNO sensors on days 1 and 3 were not statistically significant (p>0.05). The enhanced numerical accuracy afforded by rapid NO-release from sensor membranes indicates a possible advantage to greater NO fluxes, as MAP3/NO-based sensors delivered ˜3.1 μmol cm⁻² NO in <24 h. While MPTMS-RSNO sensors had a near constant MARD throughout the experiment duration, the improvements in numerical accuracy provided by lower, more sustained NO release may not have been large enough to result in improved clinical performance. Collectively, these results suggest that sensor performance benefits to a greater extent with prolonged NO release and that these gains are dependent on the fluxes at which NO is liberated.

### Biosensor Lag Time

While poor glucose sensitivity often contributes to undesirable sensor performance in vivo, diminished accuracy also results from sluggish response of the sensor to changes in BG levels. In addition to an inherent blood-tissue glucose lag, progression of the FBR increases the difficulty of glucose diffusion to the sensor. Distinct properties of the collagen capsule (e.g., thickness, density, and avascularity) produced upon resolution of the foreign body response have been shown to affect the transport properties of small molecules from the vasculature to the tissue. Even in the absence of a mature fibrotic capsule, biofouling and inflammation at the sensor-tissue interface may create a diffusion barrier to glucose. As amperometric glucose biosensors are diffusion-limited with respect to glucose, a longer response time may hinder the competence of the sensor to track rapid changes in BG levels, resulting in decreased accuracy. Since tissue surrounding NO-releasing implants exhibits less inflammation, reduced collagen encapsulation, and low impedance to glucose transport, NO-releasing sensors may show more rapid response to changes in BG. While time-shifting methods (e.g., Poincaré dynamical analysis) have been used to correct CGM data for time-lag effects, calibration of the sensor signal may corrupt a comparison of sensor lag times. Cross-correlation of the raw sensor signals and paired reference signals were thus used to estimate sensor delay, avoiding the requirement for sensor calibration.

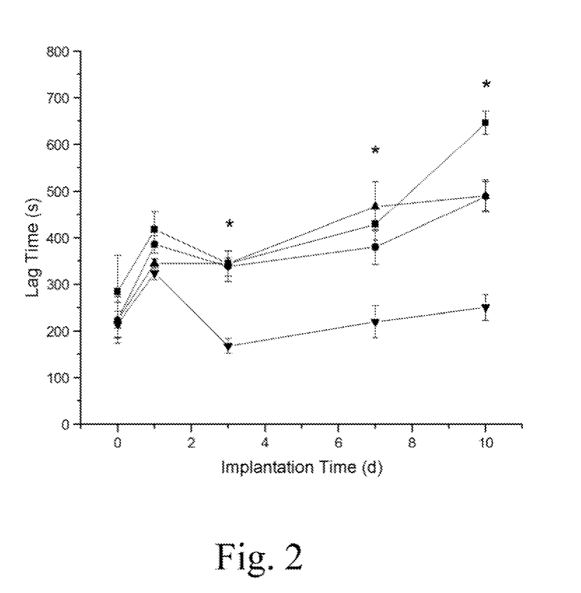

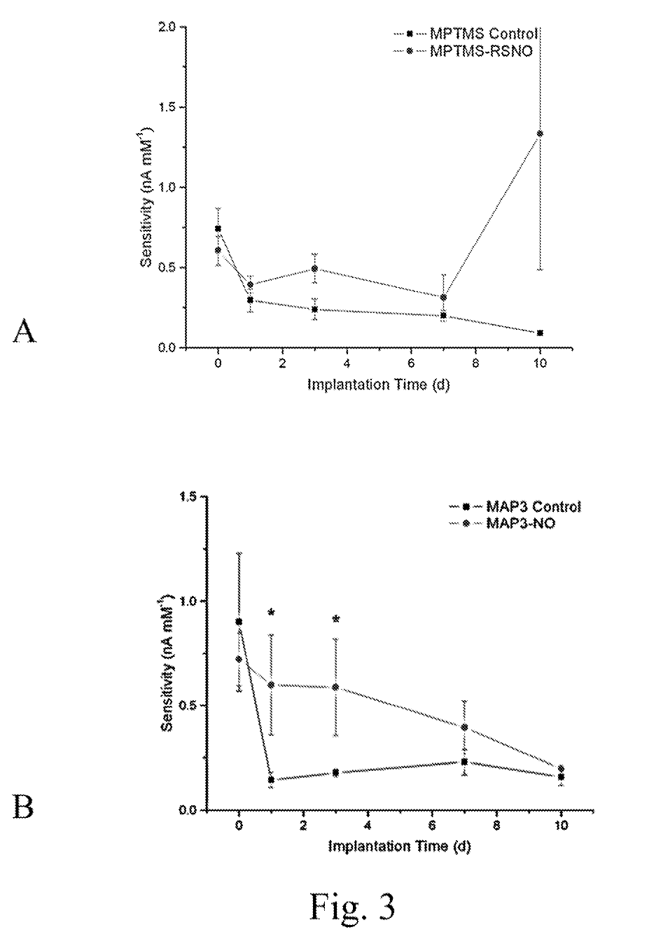

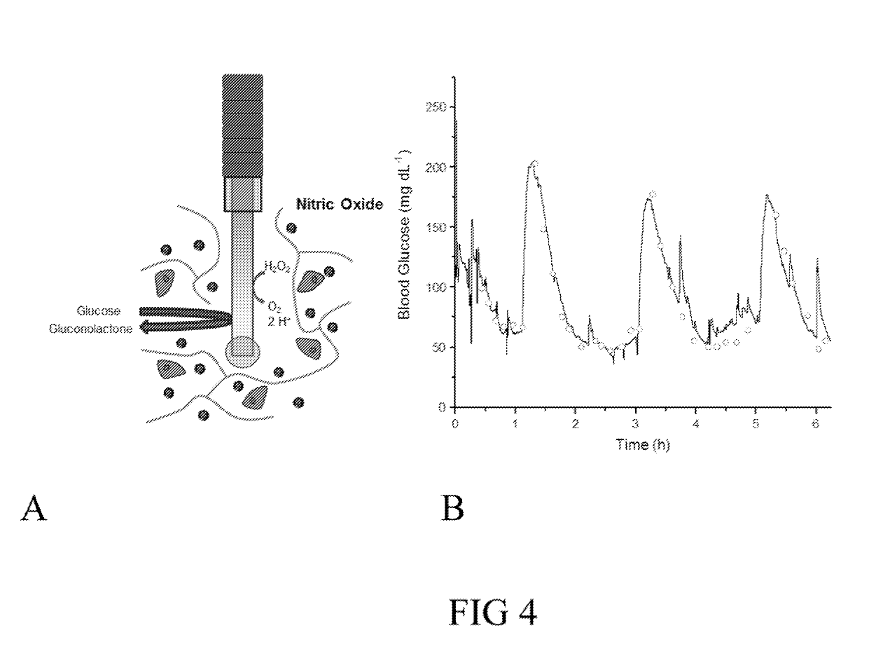

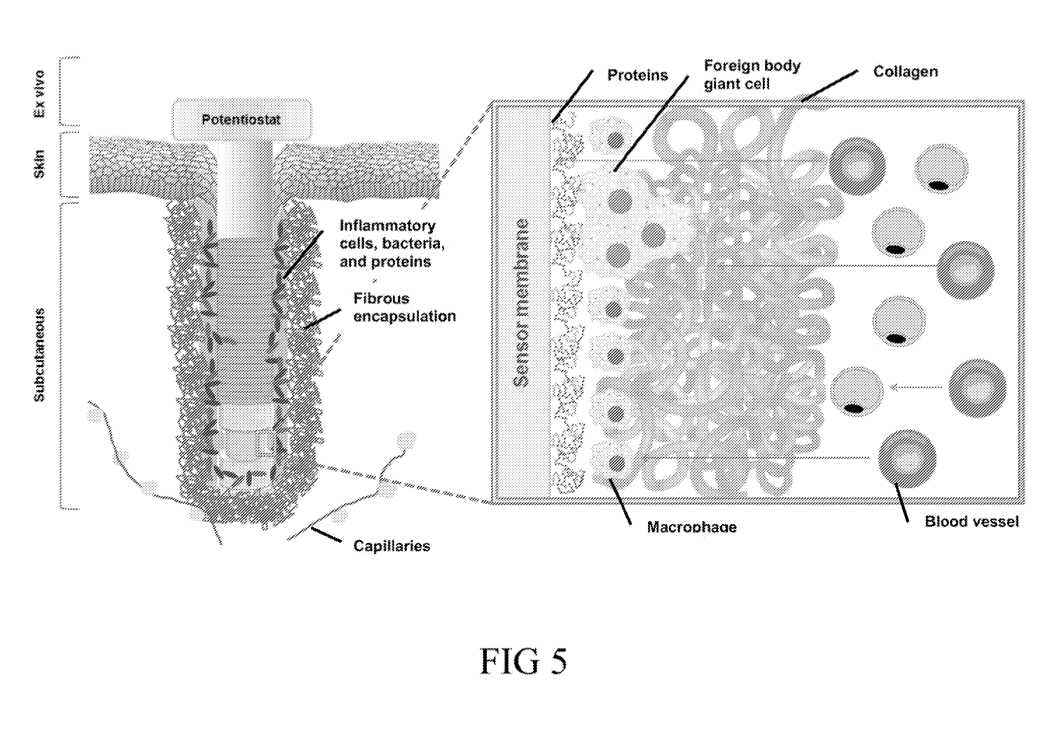

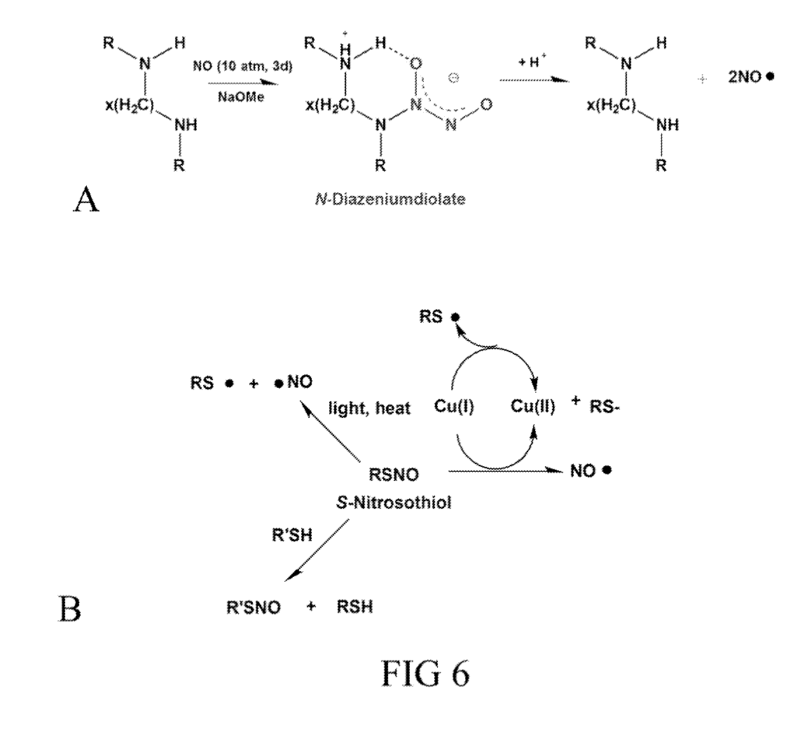

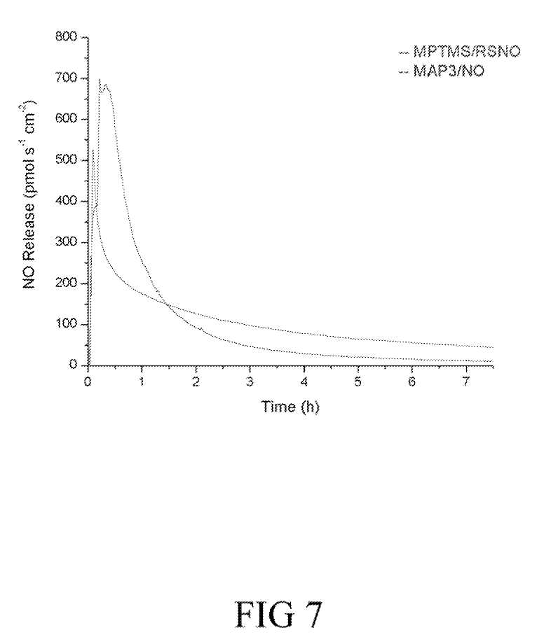

Initially (0-1 d implant period), NO release had little effect on sensor lag times (FIG. 2). However, NO release did impact sensor lag times on days 3, 7, and 10. The MPTMS-RSNO based sensors resulted in significantly faster response to changing glucose concentrations during the IVGTT (<4.2 min) compared with both control (MPTMS) and MAP3/NO-based sensors (>5.8 min). As well, the response time of the MAP3/NO-based sensors worsened with implantation time analogous to control sensors, suggesting that the benefit of reduced response time is only attained when sensors are still releasing NO. Despite similar NO payloads, the difference in lag time between the two types of NO-releasing sensors is corroborated by other work, which shows that rapid NO release at 3 and 7 d yielded no reduction in FBR, while extended NO release provided a lessened FBR at both 3 and 7 d. Likewise, sustained NO release from percutaneously implanted microdialysis probes reduced tissue impedance to glucose transport, which may explain the reduced sensor lag time observed in the present study.

Approximately 40% of implanted NO-releasing and control sensors functioned beyond 3 d. Following sensor explanation, the sensors were imaged via environmental scanning electron microscopy to investigate the implant surfaces to understand any potential sources of sensor failure. Electrical failure via membrane delamination or cracking contributed considerably to in vivo sensor failure.

Percutaneous glucose sensors, nevertheless, remain the most realistic method for implementing continuous glucose monitoring due to their low cost and facile implantation, and serve as a suitable model for evaluating candidate biomaterials Furthermore, NO is shown to provide benefits to percutaneous implants even in the presence of such physical factors.

In an embodiment, the present invention demonstrates that nitric oxide release enhances the analytical performance of in vivo glucose biosensors, with the associated benefits being dependent on the NO-release kinetics from the outer sensor membranes. Both rapid and extended NO-releasing sensors exhibited improved numerical accuracy versus controls. Rapid NO release from sensors resulted in positive differences in both clinical accuracy and glucose sensitivity, while sustained NO-release from MPTMS-RSNO biosensors provided constant numerical accuracy over long periods of time (for example, over a 10 d implant period). The MPTMS-RSNO sensors were characterized by a quicker response to the IVGTT than both the MPTMS control and MAP3-based sensors. Without being bound by theory, it is believed that the quicker response can be attributed to the generation of NO. Moreover, it is hypothesized that shorter lag times for the MPTMS-RSNO sensors are the result of improved glucose transport from the tissue surrounding the implants. The predictable performance of MPTMS-RSNO glucose biosensors suggests that materials that are capable of releasing large NO payloads for even longer durations (e.g., several weeks) represent the ultimate NO-release strategy for long-term glucose sensing technologies (e.g., months), rather than the short term (e.g., ˜10 d) period.

The following references are incorporated by reference in their entireties:
(1) Koh, A.; Nichols, S. P.; Schoenfisch, M. H. J. Diabetes Sci. Technol. 2011, 5, 1052-1059.
(2) Wilson, G. S.; Zhang, Y. In Vivo Glucose Sensing; John Wiley & Sons: Hoboken, 2009.
(3) Anderson, J. M.; Rodriguez, A.; Chang, D. T. Sem. Immunol. 2008, 20, 86-100.
(4) Gifford, R.; Kehoe, J. J.; Barnes, S. L.; Komilayev, B. A.: Alterman, M. A.; Wilson, G. S. Biomaterials 2006, 27, 2587-2598.
(5) Vroman, L.; Adams, A. L.; Fischer, G. C.; Munoz, P. C. Blood 1980, 55, 156-159.
(6) Thomé-Duret, V.; Gangnerau, M. N.; Zhang, Y.; Wilson, G. S.; Reach, G. Diabetes & Metabolism 1996, 22, 174-178.
(7) Wilson, G. S.; Ammam, M. FEBS Journal 2007, 274, 5452-5461.
(8) Nichols, S. P.; Koh, A.; Storm, W. L; Shin, J. H.; Schoenfisch, M. H. Chem. Rev. 2013, 113, 2528-2549.
(9) Ishihara, K.; Nomura, H.; Mihara, T.; Kurita, K.; Iwasaki, Y.; Nakabayashi, N. J. Biomed. Mater. Res. 1998, 39.
(10) Ju, Y. M.; Yu, B. Z.; West, L.; Moussy, Y.; Moussy, F. J. Biomed. Mater. Res. 2010, 92A.
(11) Massia, S. P.; Stark. J.; Letbetter, D. S. Biomaterials 2000, 21, 2253-2261.
(12) Sharkawy, A. A.; Klitzman, B.; Truskey, G. A.; Reichert, W. M. J. Biomed. Mater. Res. 1997, 37, 401-412.
(13) Sharkawy, A. A.; Klitzman, B.; Truskey, G. A.; Reichert, W. M. J. Biomed. Mater. Res. 1998, 40.
(14) Sharkawy, A. A.; Klitzman, B.; Truskey, G. A.; Reichert, W. M. J. Biomed. Mater. Res. 1998, 40, 598-605.
(15) Cao, H.; McHugh, K.; Chew, S. Y.; Anderson, J. M. J. Biomed. Mater. Res. 2010, 93, 1151-1159.
(16) Norton, L. W.; Tegnell, E.; Toporek, S. S.; Reichert, W. M. Biomaterials 2005, 26, 3285-3297.
(17) Ward, W. K.; Hansen, J. C.; Massoud, R. J.; Engle, J. M.; Takeno, M. M.; Hauch, K. D. J. Biomed. Mater. Res. 2010, 94, 280-287.
(18) Ziesche, E.; Scheiermann, P.; Bachmann, M.; Sadik, C. D.; Hofstetter, C.; Zwissler, B.; Pfeilschifier, J.; Muhl, H. Clin. Exp. Immunol. 2009, 157, 370-376.
(19) Klueh, U.; Dorsky, D. I.; Kreutzer, D. L. Biomaterials 2005, 26, 1155-1163.
(20) Cooke, J. P. Atheroscler. Suppl. 2003, 4, 53-60.
(21) Schwentker, A.; Vodovotz, Y.; Weller, R.; Billiar, T. R. Nitric Oxide 2002, 7, 1-10.
(22) Riccio, D. A.; Schoenfisch, M. H. Chem. Soc. Rev. 2012, 41, 3731-3741.
(23) Hetrick, E. M.; Prichard, H. L.; Klitzman, B.; Schoenfisch, M. H. Biomaterials 2007, 28, 4571-4580.
(24) Nichols, S. P.; Le, N. N.; Klitzman, B.; Schoenfisch, M. H. Anal. Chem. 2011, 83, 1180-1184.
(25) Nichols, S. P.; Koh, A.; Brown, N. L.; Rose, M. B.; Sun, B.; Slomberg, D. L.; Riccio, D. A.; Klitzman, B.; Schoenfisch, M. H. Biomaterlals 2012, 33, 6305-6312.
(26) Gifford, R.; Batchelor, M. M.; Lee, Y.; Gokulrangan, G.; Meyerhoff, M. E.; Wilson, G. S. J. Biomed. Mater. Res. 2005, 75, 755-766.
(27) Frost, M. C.; Batchelor, M. M.; Lee, Y.; Zhang, H.; Kang, Y.; Oh, B.; Wilson, G. S.; Gifford, R.; Rudich, S. M.; Meyerhoff, M. E. Microchemlcal Journal 2003, 74, 277-288.
(28) Riccio, D. A.; Nugent, J.; Schoenfisch, M. H. Chem. Mater. 2011, 23.
(29) Koh, A.; Lu, Y.; Schoenfisch, M. H. Anal. Chem. 2013, 85, 10488-10494.
(30) Koh. A.; Riccio, D. A.; Sun, B.; Carpenter, A. W.: Nichols, S. P.; Schoenfisch, M. H. Biosens. Bioelectron. 2011, 28, 17-24.
(31) Shin, J. H.; Marxer, S. M.; Schoenfisch, M. H. Anal. Chem. 2004, 76, 4543-4549.
(32) Bequette, B. W. J. Diabetes Sci. Technol. 2010, 4, 404-418.
(33) Choleau, C.; Klein, J. C.; Reach, G.; Aussedat, B.; Demaria-Pesce, V.; Wilson, G. S.; Gifford, R.; Ward, W. K. Biosens. Bloelectron. 2002, 17, 641-646.
(34) Choleau, C.; Klein, J. C.; Reach, G.; Aussedat, B.; Demaria-Pesce, V.; Wilson, G. S.; Gifford, R.; Ward, W. K. Biosens. Bioelectron. 2002, 17, 647-654.
(35) Garg, S. K.; Voelmle, M.; Gottlieb, P. A. Diabetes Res. Clin. Pract. 2010, 87, 348-353.
(36) Kovatchev, B. P.; Shields, D. S.; Breton, M. Diabetes Technol. Ther. 2009, 11, 139-143.
(37) Keenan, D. B.; Mastrototaro, J. J.; Weinzimer, S. A.; Steil, G. M. Biomed. Signal Proces. 2013, 8, 81-89.
(38) Clarke. W. L.; Kovatchev, B. P. J. Diabetes Sci. Technol. 2007, 1, 669-675.
(39) Clarke, W. L. Diabetes Technol. Ther. 2005, 7, 776-779.
(40) Venton, B. J.; Michael, D. J.; Wightman, R. M. J. Neurochem. 2003, 84, 373-381.
(41) Jacovitti, G.; Scarano, G. IEEE Trans. Signal Processing 1993, 41, 525-533.
(42) McDonald, J. H. Handbook of Biological Slatistics, 2 ed.; Sparky House Publishing: Baltimore, Md., U.S.A., 2009.
(43) Chen, S.; Jones, J. A.; Xu, Y.; Low, H. Y.; Anderson, J. M.; Leong, K. W. Biomaterials 2010, 31, 3479-3491.
(44) Barbe, C.; Bartlett, J.; Kong, L. G.; Finnie, K.; Lin, H. Q.; Larkin, M.; Calleja, S.; Bush, A.; Calleja, G. Adv. Mater. 2004, 16, 1959-1966.
(45) Shin, J. H.: Metzger, S. K.; Schoenfisch, M. H. J. Am. Chem. Soc. 2007, 129, 4612-4619.
(46) Al-Sádoni, H. H.; Ferro, A. Curr. Med. Chem. 2004, 11, 2679-2690.
(47) Vaughn, M. W.; Kuo, L.; Liao, J. C. Am. J. Physiol. 1998, 274, 2163-2176.
(48) Hetrick, E. M.; Schoenfisch, M. H. Biomaterials 2007, 28, 1948-1956.
(49) Storm, W. L.; Schoenfisch, M. H. ACS Appl. Mater. Interfaces 2013, 5, 4904-4912.
(50) Koschwanez, H. E.; Reichert, W. M. Biomaterials 2007, 28, 3687-3703.
(51) Larsen, M. O.; Rolin, B. ILAR Journal 2004, 45, 303-313.
(52) Swindle, M. M.; Makin, A.; Herron, A. J.; Clubb, F. J.; Frazier, K. S. Vet. Pathol. 2012, 49, 344-356.
(53) The Diabetes Control and Complications Trial Group N. Engl. J. Med. 1993, 329, 977-986.
(54) Wilson, G. S.; Gifford, R. Biosens. Bioelectron. 2005, 20, 2388-2403.
(55) Rebrin, K.; Fischer, U.; Hahn von Dorsche, H.; von Woetke, T.; Abel, P.; Brunstein, E. J. Biomed. Eng. 1992, 14, 33-40.
(56) Koschwanez, H. E.; Yap, F. Y.; Klitzman, B.; Reichert, W. M. J. Biomed. Mater. Res. 2008, 87, 792-807.
(57) Henry, C. Anal. Chem. 1998, 70, 594A-598A.
(58) Schaper, N. C.; Havekes, B. Diabetalogia 2012, 55, 18-20.
(59) Fahey, T. J.; Sadaty, A.; Jones, W. G.; Barber, A.; Smoller, B.; Shires. G. T. J. Surg. Res. 1991, 50, 308-313.
(60) Vinik, A. I.; Maser, R. E.; Mitchell, B. D.; Freeman, R. Diabetes Care 2003, 26, 1553-1579.
(61) Wheat, L. J. Diabetes Care 1980, 3, 187-197.
(62) U.S. Pat. No. 8,282,967.

It is contemplated and therefore within the scope of the present invention that any feature that is described above can be combined with any other feature that is described above even if those features are not discussed together. For example, the biosensor of the present invention is contemplated being used in the methods of the present invention, and the biosensor may be appropriately modified with any feature discussed above that makes the biosensor appropriately modified for that use, even if the feature is discussed in connection with a slightly different biosensor. Moreover, it should be understood that the present invention contemplates minor modifications that can be made to the biosensors and methods of the present invention without departing from the spirit and scope of the invention. Nevertheless, the invention is defined by the below claims.

We claim:

1. An implantable biosensor for determining analyte concentration levels in a subject, wherein said biosensor produces and/or releases nitric oxide at the sensor-tissue interface at a level and for a duration that allows for accurate monitoring of the analyte concentration levels in said subject; wherein said nitric oxide is liberated at a level of at least about 160 pmol/s cm² for at least about 1.5 hours in phosphate buffered saline or an equivalent biological solution.

2. The biosensor of claim 1, wherein said nitric oxide is released at a level of at least about 300 pmol/s cm² for at least about 1.5 hours.

3. The biosensor of claim 1, wherein said nitric oxide is released at a level of at least about 400 pmol/s cm² for at least about 1.5 hours.

4. The biosensor of claim 1, wherein said nitric oxide is released at a level of at least about 500 pmol/s cm² for at least about 1.5 hours.

5. The biosensor of claim 1, wherein the analyte is glucose or lactate or both.

6. The biosensor of claim 1, wherein the equivalent biological solution is tissue, interstitial fluid, blood, saline, TRIS (tris(hydroxymethyl)aminomethane) buffer, TAPS (3-[[1,3-dihydroxy-2-(hydroxymethyl)propan-2-yl]amino]propane-1-sulfonic acid) buffer, bicine (2-(Bis(2-hydroxyethyl)amino)acetic acid) buffer, Tricine (N-(2-Hydroxy-1,1-bis(hydroxymethyl)ethyl)glycine) buffer, TAPSO (3-[[1,3-dihydroxy-2-(hydroxymethyl)propan-2-yl]amino]-2-hydroxypropane-1-sulfonic acid) buffer, HEPES (2-[4-(2-hydroxyethyl)piperazin-1-yl]ethanesulfonic acid) buffer, TES (2-[[1,3-dihydroxy-2-(hydroxymethyl)propan-2-yl]amino]ethanesulfonic acid) buffer, MOPS (3-morpholinopropane-1-sulfonic acid) buffer, PIPES (1,4-Piperazinediethanesulfonic acid) buffer, cacodylate (Dimethylarsinic acid) buffer, SSC (saline sodium citrate) buffer, MES (2-(N-morpholino)ethanesulfonic acid) buffer, and succinic acid (2(R)-2-(methylamino)succinic acid) buffer.

7. A biosensor for determining glucose levels in a subject, wherein said biosensor comprises a coating that is doped with one or more macromolecular NO-donor scaffolds as a method to produce nitric oxide at the sensor-tissue interface.

8. The biosensor of claim 7, wherein the one or more macromolecular NO-donor scaffolds comprise MAP3 or MPTMS nanoparticles, or a combination of the two.

9. The biosensor of claim 8, wherein a dopant concentration is at least about 72 and 48 mg mL⁻¹ for the MAP3 and the MPTMS nanoparticles, respectively.

10. The biosensor of claim 7, wherein said nitric oxide is produced at a level of at least about 160 pmol/s cm² for at least about 1.5 hours in phosphate buffered saline or an equivalent biological solution.

11. The biosensor of claim 7, wherein one can determine glucose levels accurately using said biosensor in the subject at least about 3 days after insertion of the biosensor in said subject.

12. The biosensor of claim 7, wherein one can determine glucose levels accurately using said biosensor in the subject at least about 5 days after insertion of the biosensor in said subject.

13. The biosensor of claim 7, wherein one can determine glucose levels accurately using said biosensor in the subject at least about 7 days after insertion of the biosensor in said subject.

14. A biosensor for determining glucose levels wherein said biosensor comprises a polyurethane coating designed to release nitric oxide at a level of at least about 160 pmol/s cm² for at least about 1.5 hours in phosphate buffered saline.

15. The biosensor of claim 14, wherein the biosensor further comprises one or more of MAP3 or MPTMS nanoparticles, or combinations thereof.

16. The biosensor of claim 15, wherein the biosensor further comprises both of MAP3 and MPTMS nanoparticles.

17. A method of determining glucose concentration levels in a subject by insertion of a biosensor in said subject, the biosensor comprising a polyurethane coating that is doped with one or more of MAP3 or MPTMS nanoparticles designed to release nitric oxide, wherein the biosensor has been calibrated in a buffer to release a nitric oxide level of at least about 160 pmol/s cm² for at least about 1.5 hours.

18. The method of claim 12, wherein the biosensor is able to determine the glucose concentration 3 days after insertion of the biosensor.

19. The method of claim 12, wherein the biosensor is able to determine the glucose concentration 5 days after insertion of the biosensor.

20. The method of claim 12, wherein the biosensor is able to determine the glucose concentration 7 days after insertion of the biosensor.

21. The method of claim 12, wherein the buffer is phosphate buffered saline.
